(19) United States
(12) Patent Application Publication
(10) Pub. No.: US 2019/0262479 A1
(43) Pub. Date: Aug. 29, 2019

(54) IMAGING METHOD FOR DIFFUSE INTRINSIC PONTINE GLIOMA USING AN IMAGING AGENT, AND IMAGING AGENTS FOR EARLY STAGE DIAGNOSES

(71) Applicants: Stanley Satz, Lake Worth, FL (US); Roseanne Satz, Lake Worth, FL (US)

(72) Inventors: Stanley Satz, Lake Worth, FL (US); Roseanne Satz, Lake Worth, FL (US)

(21) Appl. No.: 16/176,658

(22) Filed: Oct. 31, 2018

Related U.S. Application Data
(60) Provisional application No. 62/580,694, filed on Nov. 2, 2017.

(51) Int. Cl. A61K 51/04

(52) U.S. Cl. CPC A61K 51/0453

(57) ABSTRACT

The present invention provides an in vivo imaging method that facilitates the diagnosis of Diffuse Intrinsic Pontine Glioma (DIPG) at an early stage. Early diagnosis is particularly advantageous as neuroprotective treatment can be applied to healthy neural cells to delay or even prevent the onset of debilitating clinical symptoms. The present invention also provides methods for producing an in vivo imaging agent useful for early diagnosis of DIPG, where embodiments of the imaging agent include a lipophilic azomycin-based hypoxic cell sensitizer labelled with an in vivo imaging moiety, and embodiments including [18F]FMISO as the lipophilic azomycin-based hypoxic cell sensitizer labelled with an in vivo imaging moiety.

### BACKGROUND OF THE INVENTION

### 1. Technical Field of the Invention

The present invention relates to in vivo imaging and in particular to an in vivo imaging method to facilitate the early diagnosis of hypoxia in pediatric Diffuse Intrinsic Pontine Glioma (DIPG).

### 2. Brief Description of the Related Art

Diffuse Intrinsic Pontine Gliomas (DIPG) are highly aggressive and difficult to treat brain tumors found at the base of the brain. They are glial tumors, meaning they arise from the brain's glial tissue—tissue made up of cells that help support and protect the brain's neurons. These tumors are found in an area of the brainstem (the lowest, stem-like part of the brain) called the pons, which controls many of the body's most vital functions such as breathing, blood pressure, and heart rate.

Diffuse Intrinsic Pontine Gliomas account for 10 percent of all childhood central nervous system (CNS) tumors. Approximately 300 children in the U.S. are diagnosed with DIPG each year. While DIPGs are usually diagnosed when children are between the ages of 5 and 9, they can occur at any age in childhood. These tumors occur in boys and girls equally and do not generally appear in adults.

When DIPGs are biopsied, they are usually grade III or grade IV. Occasionally, they are grade II, but because of their location in the brain they are still considered malignant. That being said, Diffuse Intrinsic Pontine Gliomas usually progress like grade IV glioblastoma multiforme tumors. They are very aggressive tumors and grow by invading normal brain tissue.

Diffuse Intrinsic Pontine Glioma is most commonly diagnosed from imaging studies.

Computerized tomography scan (also called a CT or CAT scan)—a diagnostic imaging procedure that uses a combination of x-rays and computer technology to produce cross-sectional images (often called slices), both horizontally and vertically, of the body. CT scans are more detailed than general x-rays.

Magnetic resonance imaging (MRI)—a diagnostic procedure that uses a combination of large magnets, radiofrequencies and a computer to produce detailed images of organs and structures within the body.

MRI provides greater anatomical detail than CT scan and does a better job of distinguishing between tumors, tumor-related swelling and normal tissue.

Magnetic resonance spectroscopy (MRS)—a diagnostic test conducted along with an MRI. It can detect the presence of organic compounds around the tumor tissue that can identify the tissue as normal or tumor, and may also be able to tell if the tumor is a glial tumor or if it is of neuronal origin (originating in a neuron, instead of an astrocytic or glial cell).

Although the above-described in vivo imaging techniques may overcome the problem of inaccurate differential diagnosis and inappropriate application of DIPG treatment, they all target the disease process at a stage when Lewy bodies (LB) and Lewy neurites (LN) are present in the CNS. LB's are abnormal aggregates that develop inside nerve cells (in Parkinson's disease), while LN's are abnormal neurites and neurons that contain granular material and abnormal α-synuclein filaments similar to those found in LB's.

### SUMMARY OF THE INVENTION

The present invention provides an in vivo imaging agent for use in a method for the diagnosis of hypoxia in pediatric Diffuse Intrinsic Pontine Glioma (DIPG) at an early stage. Early diagnosis is particularly advantageous as neuroprotective treatment can be applied to healthy neural cells to delay or even prevent the onset of debilitating clinical symptoms. A further advantage of the present invention over the prior art is that the in vivo imaging agent is provided to covalently bind to cellular molecules Therefore, it is not necessary to consider whether the in vivo imaging agent will penetrate the blood brain barrier, or to consider the relatively invasive route of direct administration of an in vivo imaging agent to the brain. The present invention also provides a method for early detection of DIPG through the administration of an in vivo imaging agent. The method comprises administering an imaging agent and detecting signals emitted based on the imaging agent interaction with cellular molecules of the subject. According to a preferred embodiment, the method may comprise administering the in vivo imaging agent intravenously. The imaging agent administered by the method preferably comprises a lipophilic azomycin-based hypoxic cell sensitizer labelled with an in vivo imaging moiety.

### BRIEF DESCRIPTION OF THE DRAWING FIGURES

FIG. 1 is a schematic diagram of an example of a dilution and trapping process used in the production of an imaging agent.

FIG. 2 is schematic diagram of an example of a process for producing an imaging agent.

FIG. 3 is a flow diagram depicting a preferred embodiment of the imaging method according to the invention.

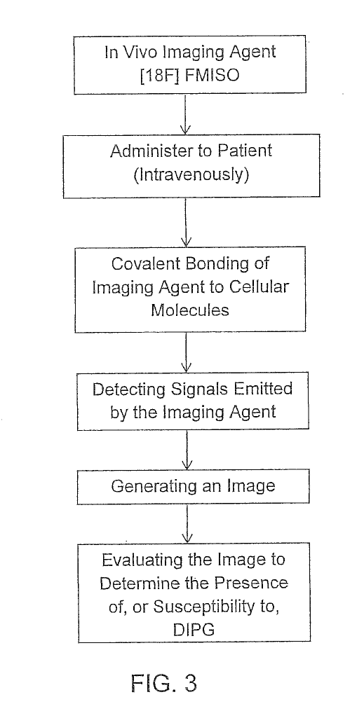

### DETAILED DESCRIPTION OF THE INVENTION

In one aspect, the present invention provides an in vivo imaging agent for use in a method to determine the presence of, or susceptibility to, hypoxia in Diffuse Intrinsic Pontine Glioma (DIPG), wherein said in vivo imaging agent comprises [18F]FMISO, ((18F) Fluoromisonidazole), a lipophilic azomycin-based hypoxic cell sensitizer labelled with an in vivo imaging moiety, that crosses the blood brain barrier and binds covalently to cellular molecules at rates that are inversely proportional to intracellular oxygen concentration levels, with oxygen levels of 3 to 10 mm Hg, said method comprising:

(i) administering to a subject a detectable quantity of said in vivo imaging agent;

(ii) allowing said administered in vivo imaging agent of step (i) to bind covalently to cellular molecules at rates that are inversely proportional to intracellular oxygen concentration levels in the autonomic nervous system (ANS) of said subject;

(iii) detecting signals emitted by said bound in vivo imaging agent of step (ii) using an in vivo imaging method;

(iv) generating an image representative of the location and/or amount of said signals; and,

(v) using the image generated in step (iv) to determine of the presence of, or susceptibility to, DIPG.

The in vivo imaging moiety is preferably chosen from: (i) a radioactive metal ion; (ii) a paramagnetic metal ion; (iii) a gamma-emitting radioactive halogen; (iv) a positron-emitting radioactive non-metal; (v) a reporter suitable for in vivo optical imaging. In vivo imaging agents may be conveniently prepared by reaction of a precursor compound with a suitable source of the in vivo imaging moiety. A “precursor compound” comprises a derivative of the in vivo imaging agent, designed so that chemical reaction with a convenient chemical form of the in vivo imaging moiety occurs site-specifically; can be conducted in the minimum number of steps (ideally a single step); and without the need for significant purification (ideally no further purification), to give the desired in vivo imaging agent. Such precursor compounds are synthetic and can conveniently be obtained in good chemical purity. The precursor compound may optionally comprise a protecting group for certain functional groups of the precursor compound.

When the in vivo imaging moiety is a radioactive metal ion, i.e. a radiometal, suitable radiometals can be either positron emitters such as ⁶⁴Cu, ⁴⁸V, ⁵²Fe, ⁵⁵Co, ^{94m}Tc or ⁶⁸Ga; or γ-emitters such as ^{99m}Tc, ¹¹¹In, ^{113m}In, or ⁶⁷Ga; and when the in vivo imaging moiety is a positron-emitting radioactive non-metal, a suitable positron-emitting radioactive non-metal may be ¹¹C, ¹³N, ¹⁵O, ¹⁷F, ¹⁸F, ⁷⁵Br, ⁷⁶Br or ¹²⁴I, with the preferred non-metal positron emitter being ¹⁸F.

When the imaging moiety comprises a metal ion, it is preferably present as a metal complex of the metal ion with a synthetic ligand. By the term “metal complex” is meant a coordination complex of the metal ion with one or more ligands. It is strongly preferred that the metal complex is “resistant to transchelation”, i.e. does not readily undergo ligand exchange with other potentially competing ligands for the metal coordination sites. Potentially competing ligands include other excipients in the preparation in vitro (e.g. radioprotectants or antimicrobial preservatives used in the preparation), or endogenous compounds in vivo (e.g. glutathione, transferrin or plasma proteins). The term “synthetic” has its conventional meaning, i.e. man-made as opposed to being isolated from natural sources e.g. from the mammalian body. Such compounds have the advantage that their manufacture and impurity profile can be fully controlled.

The method of the invention begins by administering a detectable quantity of an in vivo imaging agent to a subject. Since the ultimate purpose of the method is the provision of a diagnostically-use ml image (machine learning image), administration to the subject of said in vivo imaging agent can be understood to be a preliminary step necessary for facilitating generation of said image. In an alternative embodiment the method of the invention can be said to begin by providing a subject to whom a detectable quantity of an in vivo imaging agent has been administered. “Administering” the in vivo imaging agent means introducing the in vivo imaging agent into the subject's body, and is preferably carried out parenterally, most preferably intravenously. The intravenous route represents the most efficient way to deliver the in vivo imaging agent throughout the body of the subject. The “subject” of the invention is preferably a mammal, most preferably an intact mammalian body in vivo. In an especially preferred embodiment, the subject of the invention is a human.

The term “in vivo imaging agent” broadly refers to a compound which can be detected following its administration to the mammalian body in vivo. The in vivo imaging agent of the present invention comprises a lipophilic azomycin-based hypoxic cell sensitizer labelled with an in vivo imaging moiety. The term “labelled with an in vivo imaging moiety” means either (i) that a particular atom of the lipophilic azomycin-based hypoxic cell sensitizer is an isotopic version suitable for in vivo detection, or (ii) that a group comprising said in vivo imaging moiety is conjugated to said lipophilic azomycin-based hypoxic cell sensitizer. The in vivo imaging agent has binding affinity for α-synuclein in the range 0.1 nM-50 μM, preferably 0.1 nM-1 μM, and most preferably 0.1-100 nM. Masuda et al. (2006).

The “detection” step of the method of the invention involves the detection of signals either externally to the human body or via use of detectors designed for use in vivo, such as intravascular radiation or optical detectors such as endoscopes (e.g. suitable for detection of signals in the gut), or radiation detectors designed for intra-operative use. This detection step can also be understood as the acquisition of signal data. The “in vivo imaging method” selected for detection of signals emitted by said in vivo imaging moiety depends on the nature of the signals. Therefore, where the signals come from a paramagnetic metal ion, magnetic resonance imaging (MRI) is used, where the signals are gamma rays, single photon emission tomography (SPECT) is used, where the signals are positrons, positron emission tomography (PET) is used, and where the signals are optically active, optical imaging is used. All are suitable for use in the method of the present invention, with PET and SPECT are preferred, as they are least likely to suffer from background and therefore are the most diagnostically useful.

The in vivo imaging agent of the invention is preferably administered as a “radiopharmaceutical composition” which comprises said in vivo imaging agent, together with a biocompatible carrier, in a form suitable for mammalian administration.

The “biocompatible carrier” is a fluid, especially a liquid, in which the in vivo imaging agent as defined herein is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilize more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Such pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g., of 10 to 30 cm volume) which contains multiple patient doses, whereby single patient doses can be withdrawn into clinical grade syringes at various time intervals during the viable lifetime of the preparation to suit the clinical situation. Pre-filled syringes are designed to contain a single human dose, or “unit dose”, and are therefore preferably a disposable or other syringe suitable for clinical use.

Where the pharmaceutical composition is a radiopharmaceutical composition, the pre-filled syringe may optionally be provided with a syringe shield to protect the operator from radioactive dose. Suitable such radiopharmaceutical syringe shields are known in the art and preferably comprise either lead or tungsten.

The pharmaceutical composition may be prepared from a kit. Alternatively, it may be prepared under aseptic manufacture conditions to give the desired sterile product. The pharmaceutical composition may also be prepared under non-sterile conditions, followed by terminal sterilization using e.g. gamma-irradiation, autoclaving, dry heat or chemical treatment (e.g. with ethylene oxide).

The radiopharmaceutical composition may be prepared by a suitable method. According to some preferred embodiments, the method may comprise obtaining or generating a precursor compound, and reacting the compound to undergo a suitable labelling process where the labelling of the in vivo imaging moiety takes place.

According to an exemplary embodiment, Amino-FMISO may be synthesized, according to a proposed example, as previously described (Yukiko Masaki, Yoichi Shimizu, Takeshi Yoshioka, et al., “The accumulation mechanism of the hypoxia imaging probe “FMISO” by imaging mass spectrometry: possible involvement of low-molecular metabolites”, Scientific Reports, 19 Nov. 2015). Briefly, FMISO (25.2 mg) is dissolved in 2.5 ml methanol and 0.125 ml concentrated HCl was added. After the solution is heated to 90° C., 500 mg iron (100 mesh) is added and the mixture is refluxed for 30 min Progress of the reduction process is confirmed by the ninhydrin reaction. The reaction mixture is filtered and then purified by reversed-phase HPLC to obtain amino-FMISO (9.4 mg, 44.5%) using a Shimadzu-HPLC gradient system (LC-20AD system, Shimadzu Corporation, Kyoto, Japan) equipped with an Atlantis T3 column (250 mm×10 mm, 5 Waters Co., Milford, Mass., USA). Chromatographic separation is achieved by gradient elution with a mobile phase composed of 5 mM ammonium hydrogen carbonate (A) and acetonitrile (B). The analytes are eluted by a 1-95% B linear gradient. The total HPLC run time is proposed at 20 min at a flow rate of 4 ml/min.

The tetrahydropyranylated (THP) compound is converted into ¹⁸F-FMISO by removing the THP protecting group. This deprotection may be carried out in a reaction vessel at 90° C. by means of 1 ml of 0.6M H3PO4 for about 5 min. An acid concentration may be obtained by dilution of ˜360 μl 2.29M H₃PO₄ with ˜840 μl water.

In this exemplary embodiment, the resulting ¹⁸F-FMISO is obtained in an organic/water mixture. The organic solvent (MeCN) is removed by flushing nitrogen through right hand side connector combined with vacuum (−10 kPa (−100 mBar)) during 8 minutes at 90° C.

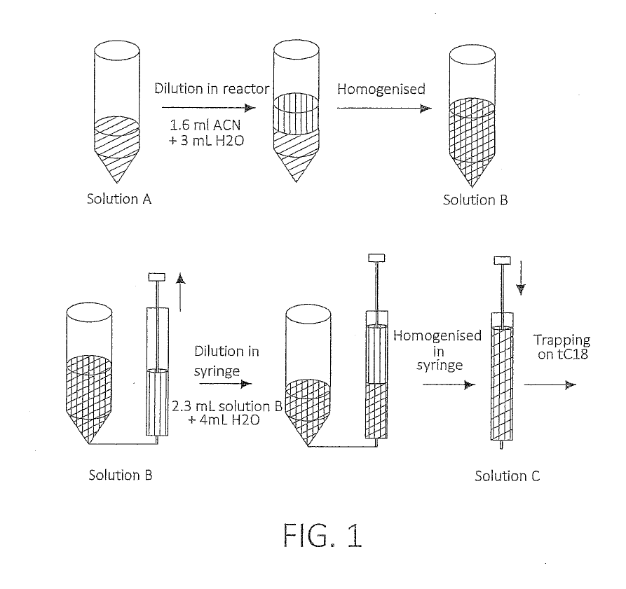

The crude FMISO is then mixed in a syringe with 3.5 ml of water, and sent back to the reaction vessel. This solution (B) is then diluted with water in 3 portions. 1.5 ml of this solution (B) is diluted with 5.0 ml of water (solution C) and then passed through the reverse phase cartridge (Oasis® HLB). This operation is done 3 times with the remaining solution in the reaction vessel. The FMISO is trapped onto the cartridge. Solvents, unreacted ¹⁸F ions and impurities are then washed off into the external waste bottle with 7 ml of water. FIG. 1 is a schematic diagram of this exemplary dilution and trapping process.

The trapped FMISO is rinsed prior the elution with a full syringe of water (˜7 ml). The elution of the FMISO is performed by dilution of absolute ethanol with water to a ratio of 5 to 6% of EtOH. This dilution is performed in the middle syringe by withdrawing ˜350 μl of EtOH first then about 6.5 ml of water and repeated 3 times. The FMISO is eluted, which in this proposed example, is from an Oasis® HLB cartridge trough an acidic alumina light cartridge to the product collection vial.

At the end of the elution, 2 full syringes of nitrogen are flushed trough the transfer tube followed by 30 sec of direct nitrogen flush (HF; 100 kPa (1000 mbar)) in order to allow a transfer trough a 15 m long tubing (min ID 1 mm). Non polar by-products are retained on the Oasis HLB cartridge and the polar, such as F18, on the alumina.

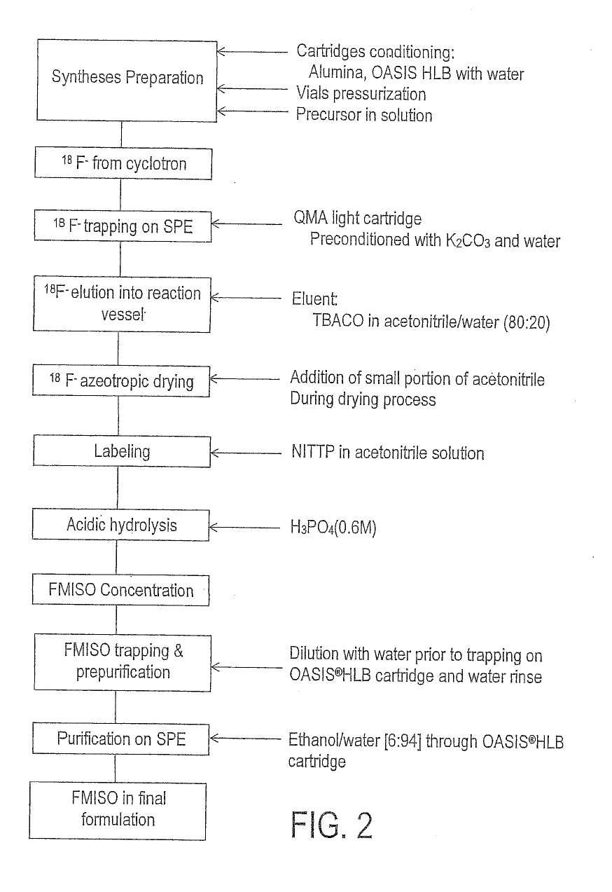

The final volume of ¹⁸F-FMISO is proposed to be about 20 mL±0.5 mL. A schematic of this exemplary process is set out in FIG. 2. The process is expected to take less than 57 minutes in total, and is anticipated to result in uncorrected yields of around 35%. An exemplary process for producing ¹⁸F-FMISO may be found in WO 2013/079578, the complete disclosure of which is incorporated by reference.

### TECHNICAL FIELD OF THE INVENTION

The present invention also relates to a method for the synthesis of 18F-labelled compounds and in particular 18F-labelled compounds that are useful as positron emission tomography (PET) tracers.

### DESCRIPTION OF RELATED ART

Hypoxia has been recognized as a significant problem in cancer of the uterine cervix. The genetic instability and molecular changes secondary to hypoxic stress promote an aggressive tumor phenotype that imparts resistance to both radiotherapy and chemotherapy, resulting in poor patient outcome. PET imaging with [F-18] fluoromisonidazole (FMISO) takes advantage of increased tracer retention in hypoxic tissues and is a non-invasive method to characterize and quantify hypoxia in cancer. Early experiences have been reported with FMISO PET as a predictor of survival in patients with cervical cancer.

The radioisotope suitable for detection in positron emission tomography (PET) have notably short half-lives. Fluorine-18 (¹⁸F) has a half-life of about 110 minutes. Synthetic methods for the production of compounds labelled with these radionuclides need to be as quick and as high yielding as possible. This is particularly important in the case of compounds destined to be used for in vivo imaging, commonly known as PET tracers. Furthermore, the step of adding the radioisotope to the compound should be as late as possible in the synthesis, and any steps taken following the addition of radioisotope for the work up and purification of the radioisotope-labelled compounds should be completed with as little time and effort as possible.

Taking [¹⁸F]FMISO, Oh et al. (2005 Nuc Med Biol; 32: 899-905) describes an automated method for its synthesis. On a TracerLab Mx [¹⁸F]FDG synthesis module (GE Healthcare) and using modified disposable [¹⁸F]FDG cassettes, a solution of the precursor compound 1-(2′-nitro--imidazolyl)-2-O-tetrahydrofuranyl-3-O-toluenesulfonyl-propanediol in acetonitrile (MeCN) was reacted with [¹⁸F] fluoride (¹⁸F) at 95-120° C. for 300-600 seconds and at 75° C. for 280 seconds, then hydrolyzed at 105° C. for 300 seconds with IN HCl following solvent removal, and neutralized using NaOH. The neutralized [¹⁸F]FMISO crude solution was purified using high-performance liquid chromatography (HPLC) to result in [¹⁸F]FMISO having decay-corrected end of synthesis (EOS) radiochemical yields of 58.5±3.5%. The reported synthesis time was 60.0±5.2 minutes. Frank et al (2009 Appl Radiat Isotop; 67(6): 1068-1070) report the synthesis of [¹⁸F]FMISO using an automated synthesizer. The precursor compound 1-(2′-nitro-1′imidazolyl)-2-O-tetrahydropyranyl-3-0-toluenesulfonyl-propanediol (NITTP) was labelled with ¹⁸F in acetonitrile at 120° C. for 10 minutes, deprotected with IN HCl at 105° C. for 5 minutes and neutralized with IN NaOH.

The neutralized crude product reaction mixture was purified using HPLC. The decay-corrected yields were reported to be 20-30%. (Id.)

The above-described automated methods for the production of [¹⁸F]FMISO both use purification by HPLC. It is preferred that a purification method taking up less time and space is used, such as solid-phase extraction (SPE). Chang et al (2007 App Rad Isotop; 65: 682-686) describe an automated method for the synthesis of [¹⁸F]FMISO using a Scanditronix Anatech RB III robotic system. The precursor compound (2′-nitro-1′-imidazolyl)-2-0-acetyl-3-0-tosylpropanol in acetonitrile was labelled with ¹⁸F at 95° C. for 10 minutes, hydrolyzed using IN HCl at 90° C. for 10 minutes following solvent removal and neutralized with a solution of NaOH. The neutralized crude reaction product was purified by first passing through a CI 8 Sep-Pak cartridge and then a neutral alumina Sep-Pak cartridge. The uncorrected EOS radiochemical yields reported were 30±5%, and the synthesis time was 65 minutes. Radiochemical yield was reduced and no apparent advantage in synthesis time was provided by this method as compared with the earlier method including HPLC purification disclosed by Oh et al (referenced above).

There is therefore scope for the provision of an automated method for the production of [¹⁸F]FMISO, and other ¹⁸F-labelled compounds wherein production comprises a hydrolytic deprotection step, that improves upon the methods known in the art.

### SUMMARY OF THE INVENTION

The present invention provides an improved method to prepare an 18F-labelled compound where the synthesis comprises a hydrolytic deprotection step. Specifically, the method of the invention permits neutralization of an acidic or basic crude product without using any neutralising chemicals. Instead, the product is trapped on an SPE column and then thoroughly rinsed with water. As a consequence of this process simplification, the method of the invention can more readily be carried out on an automated synthesizer. In addition to the radiofluorination method of the invention, the present invention provides a cassette designed to carry out the method on an automated synthesizer.

### DETAILED DESCRIPTION OF PREFERRED EMBODIMENTS

The present invention therefore provides in one aspect a method comprising: (i) labelling a protected precursor compound with F;

(ii) deprotecting the ¹⁸F-labelled compound obtained in step (i) by hydrolysis;

(iii) diluting the deprotected ¹⁸F-labelled compound obtained in step (ii) with water;

(iv) trapping the deprotected F-labelled compound on a solid-phase extraction (SPE) column by passing the diluted solution obtained in step (iii) through said column;

(v) eluting the deprotected ¹⁸F-labelled compound obtained in step (iv) from the SPE column; with the proviso that no neutralising step is carried out following the deprotection step. An “¹⁸F-labelled compound” in the context of the present invention is a chemical compound comprising at least one ¹⁸F atom. Preferably, an ¹⁸F-labelled compound of the present invention comprises only one ¹⁸F atom.

The term “labelling” in the context of the present invention refers to the radiochemical steps involved to add ¹⁸F to a compound. The precursor compound is reacted with a suitable source of ¹⁸F to result in the ¹⁸F-labelled compound. A “suitable source of ¹⁸F” is typically either ¹⁸F-fluoride or an ¹⁸F-labelled synthon. ¹⁸F-fluoride is normally obtained as an aqueous solution from the nuclear reaction ¹⁸0(p,n)¹⁸F. In order to increase its reactivity and to avoid hydroxylated by-products resulting from the presence of water, water is typically removed from ¹⁸F-fluoride prior to the reaction, and fluorination reactions are carried out using anhydrous reaction solvents (Aigbirhio et al 1995 J Fluor Chem; 70: 279-87).

The removal of water from F-fluoride is referred to as making “naked” F-fluoride. A further step that is used to improve the reactivity of ¹⁸F-fluoride for radiofluorination reactions is to add a cationic counterion prior to the removal of water. Suitably, the counterion should possess sufficient solubility within the anhydrous reaction solvent to maintain the solubility of the ¹⁸F-fluoride. Therefore, counterions that are typically used include large but soft metal ions such as rubidium or cesium, potassium complexed with a cryptand such as Kryptofix™, or tetraalkylammonium salts, wherein potassium complexed with a cryptand such as Kryptofix™, or tetraalkylammonium salts are preferred.

The term “precursor” refers to a compound that when reacted with a suitable source of a suitable source of the in vivo imaging moiety may produce the labelled imaging compound. According to preferred embodiments, the precursor may be reacted to produce an 18F-labelled imaging compound, such as, according to the preferred embodiments, ¹⁸F-FMISO.

When ¹⁸F-FMISO is the ¹⁸F-labelled compound obtained by the method of the present invention, a preferred protected precursor compound is a compound of Formula I:

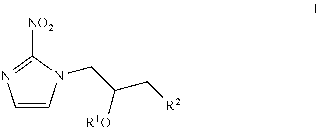

wherein:

R¹ is a protecting group for the hydroxyl function; and,

R² is a leaving group.

R¹ of Formula I is preferably selected from acetyl, benzoyl, dimethoxytrityl (DMT), β-methoxyethoxymethyl ether (MEM), methoxymethyl ether (MOM), and tetrahydropyranyl (THP), and is most preferably THP.

R² of Formula I is a leaving group, wherein the term “leaving group” refers to a moiety suitable for nucleophilic substitution and is a molecular fragment that departs with a pair of electrons in heterolytic bond cleavage. R² is preferably selected from CI, Br, I, tosylate (OTs), mesylate (OMs) and inflate (OTf), most preferably selected from OTs, OMs and OTf, and is most especially preferably OTs.

A most preferred precursor compound for the synthesis of ¹⁸F-FMISO is 1-(2′-nitro-1′-imidazolyl)-2-0-tetrahydropyranyl-3-0-tosyl-propanediol, i.e. a compound of Formula I wherein R¹ is tetrahydropyranyl and R² is OTs.

In a preferred embodiment of the invention, the diluting step comprises:

(a) adding a first volume of water to said deprotected ¹⁸F-labelled compound to obtain a first diluted solution, and,
(b) adding subsequent volumes of water to aliquots of said first diluted solution to obtain subsequent diluted solutions.

It is intended that the diluting step will result in a reaction mixture having a polarity suitable to permit high and reproducible trapping on an apolar SPE column. Ideally, the diluted reaction mixture should not have more than around 10-15% organic solvent in water in order to achieve this aim. Aliquots of the diluted solution are passed through the SPE column so as to trap the deprotected ¹⁸F-labelled compound onto the column. Optionally, once all the diluted solutions has been passed through the SPE column, an additional step of washing the column with water may be carried out prior to the eluting step.

Preferably, the eluting step is carried out using a solution of aqueous ethanol. In the case of ¹⁸F-FMISO, it is preferred that the eluting step is carried out with an aqueous ethanol solution comprising 2-20% ethanol, most preferably 5-10% ethanol. The sorbent of the SPE column for the present invention can be any silica- or polymeric-based apolar sorbent. Non-limiting examples of suitable apolar SPE columns include polymer-based Oasis HLB or Strata X SPE columns, or silica-based C2, C4, C8, CI 8, tC18 or C30 SPE columns. The SPE column of the invention is preferably selected from Oasis HLB, tCl 8, and Strata X. ¹⁸F-labelled PET tracers are now often conveniently prepared on an automated radiosynthesis apparatus. Therefore, in a preferred embodiment, the method of the present invention is an automated synthesis. The term “automated synthesis” refers to a chemical synthesis that is performed without human intervention. In other words, it refers to a process that is driven and controlled by at least one machine and that is completed without the need of manual interference.

The term “diluting” is well-known in the art and refers to the process of reducing the concentration of a solute in solution by mixing with more solvent. In the context of the present invention the solvent used in the diluting step is water. The purpose of the diluting step is to increase the polarity of the reaction mixture in order to permit high and reproducible trapping of the product on an apolar (also commonly termed “reverse-phase”) SPE column.

The term “trapping” in the present invention refers to the retention of the deprotected ^{t8}F-labelled compound on the SPE column by interactions between the deprotected ¹⁸F-labelled compound and the sorbent of the SPE column. These interactions are solvent-dependent.

The term “solid-phase extraction” (SPE) refers to the chemical separation technique that uses the affinity of solutes dissolved or suspended in a liquid (known as the mobile phase) for a solid through which the sample is passed (known as the stationary phase or sorbent) to separate a mixture into desired and undesired components. The result is that either the desired analytes of interest or undesired impurities in the sample are retained on the sorbent, i.e. the trapping step as defined above. The portion that passes through the sorbent is collected or discarded, depending on whether it contains the desired analytes or undesired impurities. If the portion retained on the sorbent includes the desired analytes, they can then be removed from the sorbent for collection in an additional step, in which the sorbent is rinsed with an appropriate eluent. The sorbent is typically packed between two porous media layers within an elongate cartridge body to form the “solid-phase extraction (SPE) column”. High-performance liquid chromatography (HPLC) is specifically excluded from the definition of SPE in the context of the present invention.

The term “neutralising” as used herein refers to the process of adjusting the pH of a solution to bring it back to pH 7, or as close as possible to pH 7. Therefore, an acidic solution can be neutralized by adding a suitable amount of an alkali such as NaOH, and an alkaline solution can be neutralized by adding a suitable amount of an acid such as HCl.

The term “eluting” refers to the process of removing the desired compound from the SPE column by passing a suitable solvent through the column. The suitable solvent for eluting is one in which the interactions between the sorbent of the SPE column and the desired compound are broken thereby allowing the compound to pass through the column and be collected.

In the method of the present invention, a distinct neutralization step is not carried out. Rather, the step of diluting serves both to bring the pH to neutrality and to prepare the reaction mixture for SPE purification. As compared to the prior art methods, the method of the present invention is therefore simplified by removal of the neutralization step, which makes the method more straightforward to carry out and to automate.

The method of the invention may be applied to the synthesis of any ¹⁸F-labelled PET tracer that comprises ¹⁸F labelling of a precursor compound that comprises protecting groups and subsequent removal of the protecting groups by acid or alkaline hydrolysis. Non-limiting examples of such ¹⁸F-labelled PET tracer include ¹⁸F-fluorodeoxyglucose (¹⁸F-FDG), 6-[¹⁸F]-L-fluorodopa (¹⁸F-FDOPA), ¹⁸F-fluoro thymidine (¹⁸F-FLT), 1-H-1-(3-[¹⁸F]fluoro-2-hydroxypropyl)-2-nitroimidazole(¹⁸F-FMISO), ¹⁸F-1-(5-fluoro-5-deoxy-a-arabinofuanosyl)-2-mitroimidazole (¹⁸F-FAZA), 16-a-[¹⁸F]-fluoroestradiol (¹⁸F-FES), and 6-[′⁸F]-fluorometarminol (¹⁸F-FMR). Said ¹⁸F-labelled compound is preferably ¹⁸F-fluorodeoxyglucose (¹⁸F-FDG), 6-[¹⁸F]-L-fluorodopa (¹⁸F-FDOPA), ¹⁸F-fluorothymidine (F-FLT), or F-fluoromisonidazole (F-FMISO), and most preferably ¹⁸F-fluorothymidine (¹⁸F-FLT) or ¹⁸F-fluoromisonidazole (¹⁸F-FMISO). The known synthesis of each of these PET tracers includes a deprotection step and a neutralization step (see for example chapters 6 and 9 of “Handbook of Radiopharmaceuticals” 2003; Wiley: by Welch and Redvanly, and chapter 8 of “Basics of PET Imaging, 2^{nd} Edition” 2010; Springer: by Saha). The method of the invention is carried out to obtain any of these PET tracers in purified form in a straightforward manner by omitting the neutralization step and carrying out the diluting, trapping and eluting steps as defined herein. Examples of PET tracers which may be synthesized by the method of this aspect of the present invention include [¹⁸F]-fluorodeoxyglucose ([¹⁸F]-FDG), [¹⁸F]-fluorodihydroxyphenylalanine ([¹⁸F]F-DOPA), [¹⁸F]-fluorouracil, [¹⁸F]-1-amino-3-fluorocyclobutane-1-carboxylic acid ([¹⁸F]-FACBC), [′⁸F]-altanserine, [¹⁸F]-fluorodopamine, 3′-deoxy-3′-¹⁸F-fluorothymidine [¹⁸F-FLT] and [¹⁸F]-fluorobenzothiazoles.

The structures of various ¹⁸F-labelled protected precursor compounds obtained in step (i) of the method of the present invention are as follows (wherein P¹ to P⁴ are each independently hydrogen or a protecting group):

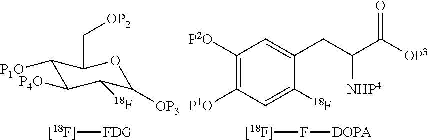

In one embodiment, the method of the invention is used for the synthesis of F-FMISO:

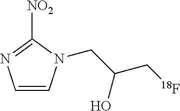 [SMILES: O=[N+]([O-])c1nccn1CC(O)C[18F]]

There are several commercially-available examples of such apparatus, including Tracerlab™ and Fastlab™ (GE Healthcare Ltd). Such apparatus commonly comprises a “cassette”, often disposable, in which the radiochemistry is performed, which is fitted to the apparatus in order to perform a radiosynthesis. The cassette normally includes fluid pathways, a reaction vessel, and ports for receiving reagent vials as well as any solid-phase extraction cartridges used in post-radiosynthetic clean up steps. The automation of synthesis of PET tracers performed on a synthesiser platform is limited by the number of available reagent slots. The method of the present invention permits a reduction in the number of chemicals required by removing the neutralising agent. In another aspect, the present invention provides a cassette for carrying out the method of the invention, said cassette comprising:

(i) a vessel containing said protected precursor compound as defined herein;

(ii) means for eluting the vessel containing said protected precursor compound with a suitable source of F as defined herein;

(iii) means for deprotecting the ¹⁸F-labelled compound obtained following elution of the vessel containing said protected precursor compound with a suitable source of ¹⁸F; and,

(iv) an SPE column as defined herein suitable for trapping the deprotected ¹⁸F-labelled compound; with the proviso that a vessel containing a neutralization agent suitable for neutralizing the pH of said deprotected ¹⁸F-labelled compound is neither comprised in or in fluid connection with said cassette.

In the context of the cassette of the invention, a “neutralizing agent” is an acidic or an alkaline solution designed to neutralize the pH of, respectively an alkaline or an acidic solution comprising deprotected labelled ¹⁸F-labelled compound.

All the suitable, preferred, most preferred, especially preferred and most especially preferred embodiments of the precursor compound of Formula I, ¹⁸F-fluoride and the SPE cartridges that are presented herein in respect of the method of the invention also apply to the cassette of the invention.

The cassette of the invention may furthermore comprise:

(iv) an ion-exchange cartridge for removal of excess [¹⁸F]-fluoride.

### BRIEF DESCRIPTION OF THE EXAMPLES

Example 1 describes how ¹⁸F-FMISO was obtained according to the method of the invention.

### List of Abbreviations Used in the Examples

EtOH ethanol;

¹⁸F fluoride;

¹⁸F-FMISO 1-H-1-(3-[¹⁸F]fluoro-2-hydroxypropyl)-2-nitroimidazole;

ID internal diameter;

NITTP 1-(2′-Nitro-1′-imidazolyl)-2-0-tetrahydropyranyl-3-0-toluenesulfonyl-propanediol;

MeCN acetonitrile;

THP tetrahydropyranyl.

### Diagnosis and Treatment Monitoring

¹⁸F-fluoromisonidazole (FMISO) has been widely used as a hypoxia imaging probe for diagnostic positron emission tomography (PET). As reported by Masaki, Y. et al. “The accumulation mechanism of the hypoxia imaging probe “FMISO” by imaging mass spectrometry: possible involvement of low-molecular metabolites” 5, 16802; doi: 10.1038/srep16802 (2015), FMISO is believed to accumulate in hypoxic cells via covalent binding with macromolecules after reduction of its nitro group. However, its detailed accumulation mechanism remains unknown. Therefore, what was investigated were the chemical forms of FMISO and their distributions in tumors using imaging mass spectrometry (IMS), which visualizes spatial distribution of chemical compositions based on molecular masses in tissue sections. A radiochemical analysis revealed that most of the radioactivity in tumors existed as low-molecular-weight compounds with unknown chemical formulas, unlike observations made with conventional views, suggesting that the radioactivity distribution primarily reflected that of these unknown substances. An IMS analysis indicated that FMISO and its reductive metabolites were nonspecifically distributed in the tumors in patterns not corresponding to the radioactivity distribution.

1-27. (canceled)

28. A method for determining the presence of, or susceptibility to, Diffuse Intrinsic Pontine Glioma (DIPG) in a mammalian subject, wherein said in vivo imaging agent comprises a compound labelled with an in vivo imaging moiety having a binding affinity for α-synuclein, the method comprising the steps of:
(i) administering to a subject a detectable quantity of said in vivo imaging agent;
(ii) allowing said administered in vivo imaging agent of step (i) to bind to α-synuclein deposits in the autonomic nervous system (ANS) of said subject;
(iii) detecting signals emitted by said bound in vivo imaging agent of step (ii) using an in vivo imaging method;
(iv) generating an image representative of the location and/or amount of said signals; and,
(v) using the image generated in step (iv) to determine of the presence of, or susceptibility to, Diffuse Intrinsic Pontine Glioma (DIPG).

29. The method of claim 28, wherein said compound labelled with an in vivo imaging moiety having a binding affinity for α-synuclein comprises a lipophilic azomycin-based hypoxic cell sensitizer labelled with the in vivo imaging moiety.

30. The method of claim 29, wherein said lipophilic azomycin-based hypoxic cell sensitizer comprises an isotopic version capable of being detected in vivo, and wherein detecting signals emitted by said bound in vivo imaging agent of step (ii), using an in vivo imaging method, comprises detecting said isotopic version.

31. The method of claim 30, wherein at least one atom of the lipophilic azomycin-based hypoxic cell sensitizer comprises an isotopic version capable of being detected in vivo, and wherein detecting signals emitted by said bound in vivo imaging agent of step (ii), using an in vivo imaging method, comprises detecting signals emitted by from said at least one atom of said isotopic version of said lipophilic azomycin-based hypoxic cell sensitizer.

32. The method of claim 29, wherein either:
(a) a particular atom of the lipophilic azomycin-based hypoxic cell sensitizer is an isotopic version suitable for in vivo detection, and wherein detecting signals emitted by said bound in vivo imaging agent of step (ii) using an in vivo imaging method comprises detecting signals emitted by the isotopic version of the lipophilic azomycin-based hypoxic cell sensitizer; or
(b) a group comprising said in vivo imaging moiety is conjugated to said lipophilic azomycin-based hypoxic cell sensitizer, and wherein detecting signals emitted by said bound in vivo imaging agent of step (ii) using an in vivo imaging method comprises detecting signals emitted by the vivo imaging moiety is conjugated to said lipophilic azomycin-based hypoxic cell sensitizer.

33. The method of claim 28, wherein said lipophilic azomycin-based hypoxic cell sensitizer labelled with the in vivo imaging moiety having the binding affinity for α-synuclein includes at least one ¹⁸F atom.

34. The method of claim 33, wherein said lipophilic azomycin-based hypoxic cell sensitizer labelled with the in vivo imaging moiety having the binding affinity for α-synuclein comprises [18F]FMISO.

35. The method of claim 29, wherein the in vivo imaging agent has binding affinity for α-synuclein in the range 0.1 nM-50 μM.

36. The method of claim 35, wherein the in vivo imaging agent has binding affinity for α-synuclein in the range of 0.1 nM-1 μM.

37. The method of claim 36, wherein the in vivo imaging agent has binding affinity for α-synuclein in the range of 0.1-100 nM.

38. The method of claim 29, wherein said lipophilic azomycin-based hypoxic cell sensitizer labelled with an in vivo imaging moiety comprises [18F]FMISO.

39. The method of claim 38, wherein said lipophilic azomycin-based hypoxic cell sensitizer labelled with an in vivo imaging moiety crosses the blood brain barrier of said mammalian subject during step (ii).

40. The method of claim 39, wherein said lipophilic azomycin-based hypoxic cell sensitizer labelled with an in vivo imaging moiety binds covalently to cellular molecules at rates that are inversely proportional to intracellular oxygen concentration levels.

41. The method of claim 40, wherein said lipophilic azomycin-based hypoxic cell sensitizer labelled with an in vivo imaging moiety binds covalently to cellular molecules at rates that are inversely proportional to intracellular oxygen concentration levels, wherein said oxygen levels are 3 to 10 mm Hg.

42. The method of claim 28, wherein said in vivo imaging moiety is selected from:
(i) a radioactive metal ion;
(ii) a paramagnetic metal ion;
(iii) a gamma-emitting radioactive halogen;
(iv) a positron-emitting radioactive non-metal,
(v) a reporter suitable for in vivo optical imaging.

43. The method of claim 42, wherein said positron-emitting radioactive non-metal comprises 18F, and wherein said lipophilic azomycin-based hypoxic cell sensitizer labelled with an in vivo imaging moiety having a binding affinity for α-synuclein comprises [18F]FMISO.

44. The method of claim 29, further comprising producing the in vivo imaging agent comprising the lipophilic azomycin-based hypoxic cell sensitizer labelled with the in vivo imaging moiety having the binding affinity for α-synuclein, wherein producing comprises the steps of:
(i) labelling a protected precursor compound with 18F;
(ii) deprotecting the 18F-labelled compound obtained in step (i) by hydrolysis;
(iii) diluting the deprotected 18F-labelled compound obtained in step (ii) with water;
(iv) trapping the deprotected 18F-labelled compound on a solid-phase extraction (SPE) column by passing the diluted solution obtained in step (iii) through said column;
(v) eluting the deprotected 18F-labelled compound from the SPE column;
with the proviso that no neutralising step is carried out following the deprotection step.

45. The method of claim 44, wherein said deprotecting step (ii) is carried out by acid hydrolysis.

46. The method of claim 44, wherein said 18F-labelled compound is a compound selected from the group consisting of: 18F-fluoromisonidazole (18F-FMISO); and 1-H-1-(3-[18F]fluoro-2-hydroxypropyl)-2-nitroimidazole (18F-FMISO).

47. The method of claim 44, which is automated.
